(19) United States
(12) Patent Application Publication
(10) Pub. No.: US 2024/0331153 A1
(43) Pub. Date: Oct. 3, 2024

(54) DYNAMIC IMAGE ANALYSIS APPARATUS, RECORDING MEDIUM, AND DYNAMIC IMAGE ANALYSIS METHOD

(71) Applicant: Konica Minolta, Inc., Tokyo (JP)

(72) Inventor: Noritsugu MATSUTANI, Tokyo (JP)

(21) Appl. No.: 18/622,697

(22) Filed: Mar. 29, 2024

(30) Foreign Application Priority Data
Apr. 3, 2023 (JP) 2023-060054

(51) Int. Cl. G06T 7/00; A61B 6/00

(52) U.S. Cl. CPC G06T 7/0012; A61B 6/5258; G06T 2200/28; G06T 2207/10116; G06T 2207/20092; G06T 2207/30061; G06T 2207/30104

(57) ABSTRACT

A dynamic image analysis apparatus includes a hardware processor. The hardware processor, acquires a dynamic image obtained by radiographing a dynamic state of a subject, analyzes the dynamic image using one or more types of analysis parameters, generates an analysis result image, determines whether the analysis parameter needs to be corrected, and notifies the analysis parameter determined to require correction.

### CROSS REFERENCE TO RELATED APPLICATIONS

The entire disclosure of Japanese Patent Application No. 2023-060054, filed on Apr. 3, 2023, including description, claims, drawings and abstract is incorporated herein by reference in its entirety.

### BACKGROUND OF THE INVENTION

### Technical Field

The present invention relates to a dynamic image analysis device, a recording medium, and a dynamic image analysis method.

### Description of Related Art

Conventionally, dynamic images have been used for diagnosis. The dynamic image is an image obtained by capturing a dynamic state of a subject by radiography. In the dynamic image, it is possible to capture the dynamic state of the subject which cannot be captured in the still image. Therefore, it is possible to acquire motion information and functional information of the subject by analyzing the dynamic image in the dynamic image analysis apparatus. For example, by performing functional analysis such as blood flow analysis or ventilation analysis on the dynamic image of the chest by the dynamic image analysis apparatus, an analysis result image indicating functional information of the chest, for example, blood flow information or ventilation information can be provided for diagnosis.

In order to provide the analysis result image for diagnosis, a user such as a technician needs to determine whether the analysis result image has quality that can be used for diagnosis. However, this determination requires skill. Therefore, for example. Japanese Unexamined Patent Publication No. 2022-103679 describes a dynamic image analysis apparatus that determines whether a feature amount related to dynamic analysis obtained from an analysis result of a dynamic image satisfies a criterion to determine whether the analysis result can be used for diagnosis, and displays the determination result.

The dynamic image analysis apparatus described in Japanese Unexamined Patent Publication No. 2022-103679 merely displays a determination result as to whether the analysis result can be used for diagnosis. Therefore, in a case where the determination result shows that the analysis result cannot be used for diagnosis, the user needs to perform imaging again. However, it may be possible to obtain an analysis result image suitable for diagnosis by correcting analysis parameters used for analysis without re-imaging. However, it is difficult for the user to recognize which analysis parameter should be corrected.

An object of the present invention is to enable a user to easily recognize an analysis parameter that needs to be corrected among analysis parameters for analyzing a dynamic image.

### SUMMARY OF THE INVENTION

In order to solve the above problem, a dynamic image analysis apparatus includes: a hardware processor, wherein the hardware processor, acquires a dynamic image obtained by radiographing a dynamic state of a subject, analyzes the dynamic image using one or more types of analysis parameters, generates an analysis result image, determines whether the analysis parameter needs to be corrected, and notifies the analysis parameter determined to require correction.

Furthermore, a non-transitory computer-readable recording medium including a program causes a computer used in a dynamic image analysis apparatus to, acquire a dynamic image obtained by radiographing a dynamic state of a subject, analyze the dynamic image using one or more types of analysis parameters, generate an analysis result image, determine whether the analysis parameter needs to be corrected, and notify the analysis parameter determined to require correction.

In addition, the dynamic image analysis method of the present invention includes, acquiring a dynamic image obtained by radiographing a dynamic state of a subject: analyzing the dynamic image using one or more types of analysis parameters: generating an analysis result image: determining whether the analysis parameter needs to be corrected; and notifying the analysis parameter determined to require correction.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a diagram illustrating an overall configuration of a dynamic analysis system in an embodiment of the present invention;

FIG. 2 is a diagram illustrating an example of an analysis parameter table when the type of analysis processing is blood flow analysis:

FIG. 3 is a diagram illustrating an example of an analysis parameter table when the type of analysis processing is ventilation analysis:

FIG. 4 is a diagram illustrating an example of a cause identification table when the type of analysis processing is blood flow analysis:

FIG. 5 is a flowchart illustrating imaging control processing executed by a controller of an imaging console of FIG. 1:

FIG. 6 is a flowchart illustrating dynamic analysis processing A executed by the controller of the analyzing console shown in FIG. 1 according to the first embodiment:

FIG. 7 is a diagram illustrating a cardiac ROI, a direct ROI, an aortic arch ROI, an aortic root ROI, and a cardiac dimension which are set on a dynamic image:

FIG. 8 is a diagram showing an analysis segment and a reference frame image on a graph representing a temporal change in a mean concentration in the cardiac ROI:

FIG. 9 is a diagram illustrating an analysis segment and a reference frame image on a graph indicating a cardiac dimension, a heart area, a mean concentration in an aortic arch ROI, and a mean concentration in an aortic root ROI.:

FIG. 10 is a diagram illustrating an example of an analysis parameter correction notification screen:

FIG. 11 is a flowchart illustrating dynamic analysis processing B executed by the controller of the analyzing console shown in FIG. 1 according to the second embodiment; and

FIG. 12 is a diagram showing an example of a reliability notification screen displayed in step S49 of FIG. 11.

### DETAILED DESCRIPTION

Hereinafter, one or more embodiments of the present invention will be described with reference to the drawings. However, the scope of the invention is not limited to the disclosed embodiments.

In the following, embodiments of the present invention will be described with reference to the drawings. However, the scope of the invention is not limited to the illustrated examples.

### First Embodiment

### [Configuration of Dynamic Analysis System 100]

First, a configuration of the present embodiment will be described.

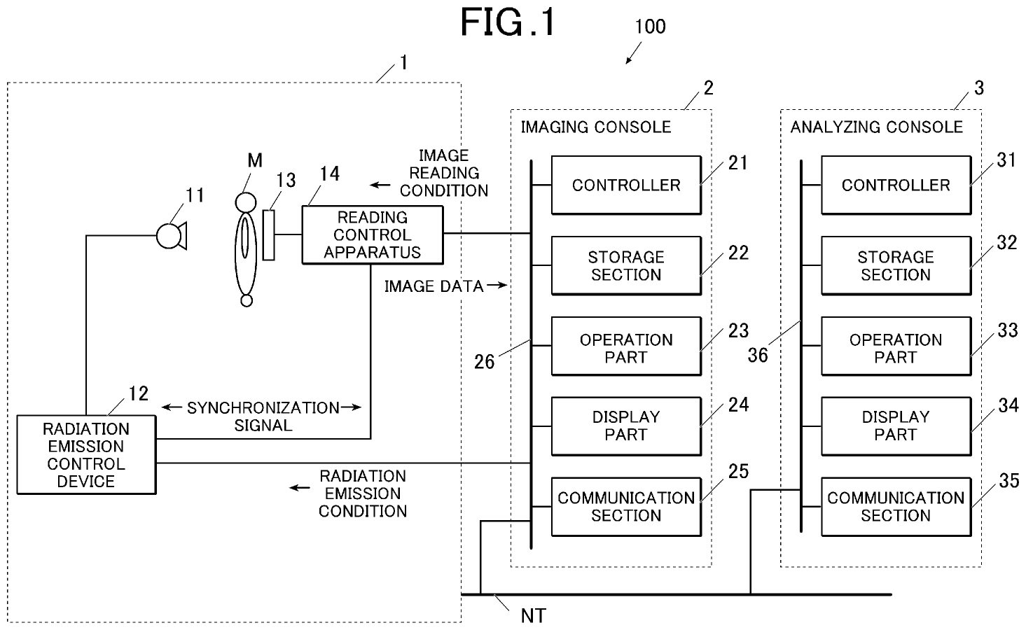

FIG. 1 is a diagram illustrating an example of the entire structure of a dynamic analysis system 100 in the present embodiment.

As illustrated in FIG. 1, the dynamic analysis system 100 includes an imaging apparatus 1, an imaging console 2, and an analyzing console 3. The imaging apparatus 1 and the imaging console 2 are connected to each other by a communication cable or the like. The imaging console 2 and the analyzing console 3 are connected to each other via a communication network NT such as a local area network (LAN). The apparatuses included in the dynamic analysis system 100 comply with the Digital Image and Communications in Medicine (DICOM) standard. Communication between the above-described apparatuses is performed in accordance with DICOM.

### [Configuration of Imaging Apparatus 1]

The imaging apparatus 1 is, for example, an imaging unit that performs radiation imaging of a dynamic state of a chest having periodicity: such as a change in form of a lung expanding and contracting due to respiratory motion and pulsation of a heart. The dynamic imaging refers to acquiring a plurality of images indicating a dynamic state of a subject by repeatedly irradiating the subject with pulsed radiation such as X-rays at predetermined time intervals (pulse emission) or continuously irradiating the subject with radiation at a low dose rate (continuous emission). A series of images obtained by dynamic imaging is referred to as dynamic images. Each of the plurality of images constituting the dynamic image is referred to as a frame image.

Here, the dynamic imaging includes moving image capturing, but does not include capturing a still image while displaying a moving image. The dynamic image includes a moving image, but does not include an image obtained by capturing a still image while displaying a moving image.

In the following embodiment, a case where dynamic imaging of the front of the chest is performed by pulse emission will be described.

The radiation source 11 is arranged at a position facing the radiation detection section 13 with the subject M interposed therebetween. The radiation source 11 irradiates the subject M with radiation (X-rays) under the control of the radiation emission control device 12.

The radiation emission control device 12 is connected to the imaging console 2. The radiation emission control device 12 performs radiation imaging by controlling the radiation source 11 based on the radiation emission conditions input from the imaging console 2. The radiation emission conditions input from the imaging console 2 include, for example, a pulse rate, a pulse width, a pulse interval, the number of imaging frames per imaging, a value of an X-ray tube current, a value of an X-ray tube voltage, and a type of additional filter. The pulse rate is the number of times of radiation emission per second, and matches a frame rate to be described later. The pulse width is a radiation emission time per radiation emission. The pulse interval is a time from the start of one radiation emission to the start of the next radiation emission. The pulse interval coincides with a frame interval which will be described later.

The radiation detection section 13 includes a semiconductor image sensor, such as a flat panel detector (FPD). The FPD includes, for example, a glass substrate or the like. In the FPD, a plurality of detection elements (pixels) are arranged in a matrix at predetermined positions on a glass substrate. The plurality of detection elements detect radiation emitted from the radiation source 11 and transmitted through at least the subject M in accordance with the intensity of the radiation, convert the detected radiation into an electric signal, and accumulate the electric signal. Each pixel includes a switching section such as a thin film transistor (TFT). There are an indirect conversion type FPD and a direct conversion type FPD, and either one of them may be used as the radiation detection section 13. The indirect conversion type converts X-rays into electric signals by photoelectric conversion elements via a scintillator. The direct conversion type converts X-rays directly into electric signals. The radiation detection section 13 faces the radiation source 11 with the subject M interposed therebetween.

The reading control apparatus 14 is connected to the imaging console 2. The reading control apparatus 14 controls the switching section of each pixel of the radiation detection section 13 on the basis of image reading conditions input from the imaging console 2. The reading control apparatus 14 switches reading of the electric signal accumulated in each pixel, and acquires image data by reading the electric signal accumulated in the radiation detection section 13. This image data is a frame image. Then, the reading control apparatus 14 outputs the acquired frame image to the imaging console 2. The image reading condition is, for example, a frame rate, a frame interval, a pixel size, an image size (matrix size), or the like. The frame rate is the number of frame images acquired per second. The frame rate coincides with the pulse rate. The frame interval is the time from the start of the operation of acquiring one frame image to the start of the operation of acquiring the next frame image. The frame interval coincides with the pulse interval.

Here, the radiation emission control device 12 and the reading control apparatus 14 are connected to each other, and exchange synchronization signals with each other to synchronize the radiation emission operation and the image reading operation.

### [Configuration of Imaging Console 2]

The imaging console 2 outputs radiation emission conditions and image reading conditions to the imaging apparatus 1 to control the radiography and the reading of the radiation image by the imaging apparatus 1. In addition, the imaging console 2 displays the dynamic image acquired by the imaging apparatus 1 for confirmation of positioning and confirmation of whether the image is suitable for diagnosis by a photographer such as a radiographer.

As illustrated in FIG. 1, the imaging console 2 includes a controller 21 (hardware processor), a storage section 22, an operation part 23, a display part 24, and a communication section 25. These components of the imaging console 2 are connected to each other by a bus 26.

The controller 21 includes a central processing unit (CPU), a random access memory (RAM), and the like. In response to an operation of the operation part 23, the CPU of the controller 21 reads a system program and various processing programs stored in the storage section 22 and loads them into the RAM. The CPU of the controller 21 centrally controls the operation of each section of the imaging console 2 and the radiation emission operation and the reading operation of the imaging apparatus 1 by executing various processing including imaging control processing, which will be described later, according to the expanded program.

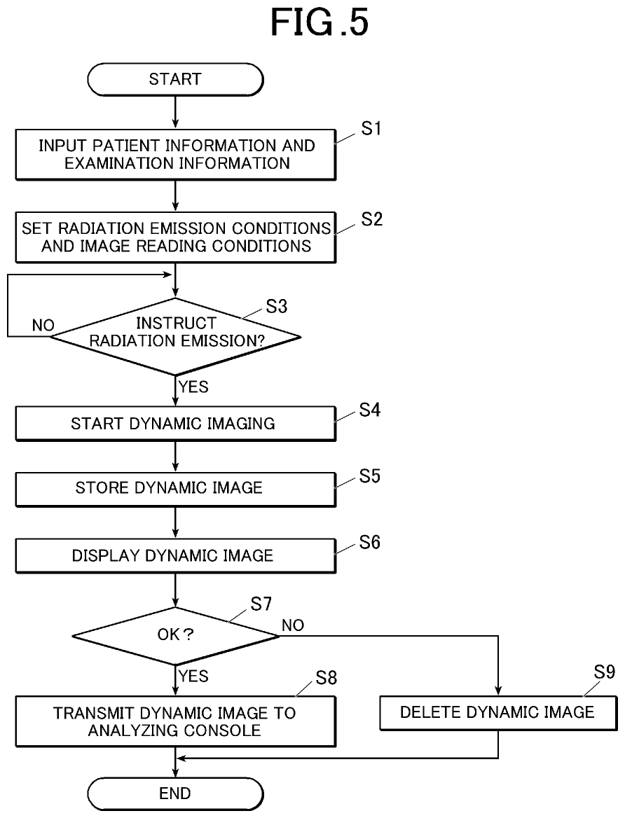

The storage section 22 includes a nonvolatile semiconductor memory, a hard disk and the like. The storage section 22 stores various programs to be executed by the controller 21, parameters required for executing processing by the programs, and data such as processing results. For example, the storage section 22 stores a program for executing the imaging control process shown in FIG. 5. Further, the storage section 22 stores the radiation emission condition and the image reading condition in association with the examination target site and the imaging direction. The various programs are stored in the form of readable program codes. The controller 21 sequentially performs operations in accordance with the program codes.

The operation part 23 includes a keyboard including cursor keys, numeric input keys, various function keys, and the like, and a pointing device such as a mouse. The operation part 23 outputs, to the controller 21, an instruction signal input by a user's key operation on the keyboard or a user's mouse operation. Furthermore, the operation part 23 may include a touch screen on the display screen of the display part 24. In this case, the operation part 23 outputs an instruction signal input via the touch screen to the controller 21.

The display part 24 includes a monitor such as a liquid crystal display (LCD) or a cathode ray tube (CRT). The display part 24 displays an input instruction from the operation part 23, data, and the like in accordance with an instruction of a display signal input from the controller 21.

The communication section 25 includes a LAN adapter, a modem, or a terminal adapter (TA). The communication section 25 controls data transmission and reception to and from each device connected to the communication network NT.

### [Configuration of Analyzing Console 3]

The analyzing console 3 is a dynamic image analyzing apparatus that acquires the dynamic image of the chest from the imaging console 2 and generates an analysis result image by analyzing the acquired dynamic image. The analyzing console 3 can transmit and receive data to and from an image server of a PACS (Picture Archiving and Communication System) (not shown) via the communication network NT.

As illustrated in FIG. 1, the analyzing console 3 includes a controller 31 (hardware processor), a storage section 32, an operation part 33, a display part 34, and a communication section 35. The respective units of the analyzing console 3 are connected by a bus 36.

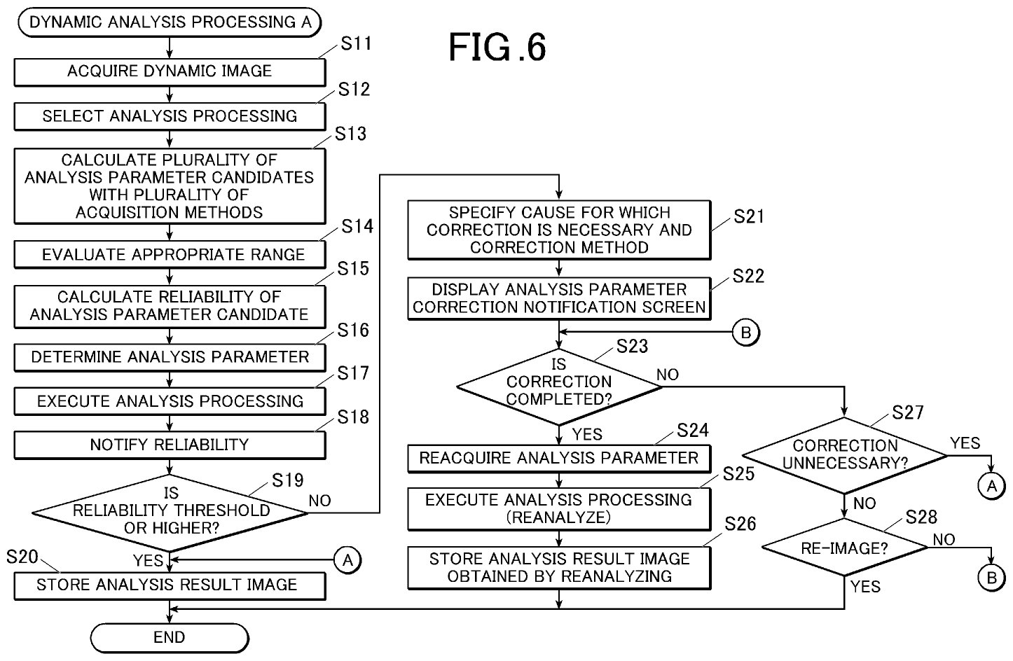

The controller 31 includes a CPU, a RAM, and the like. In response to an operation of the operation part 33, the CPU of the controller 31 reads a system program or various processing programs stored in the storage section 32 and loads the program into the RAM. The CPU of the controller 31 executes various processes including a dynamic analysis process A (see FIG. 6) to be described later in accordance with the expanded program. The controller 31 functions as an acquisition unit, an analysis processing unit, a determination unit, a notification unit, a reliability calculation unit, a decision unit, a correction input unit, and a correction unit of the present invention. The storage section 32 includes a nonvolatile semiconductor memory, a hard disk and the like. The storage section 32 stores various programs, parameters necessary for executing processing by the programs, and data such as processing results. The programs stored in the storage section 32 include a program for executing the dynamic analysis processing A in the controller 31. These various programs are stored in the form of readable program codes. The controller 31 sequentially executes operations according to the program code.

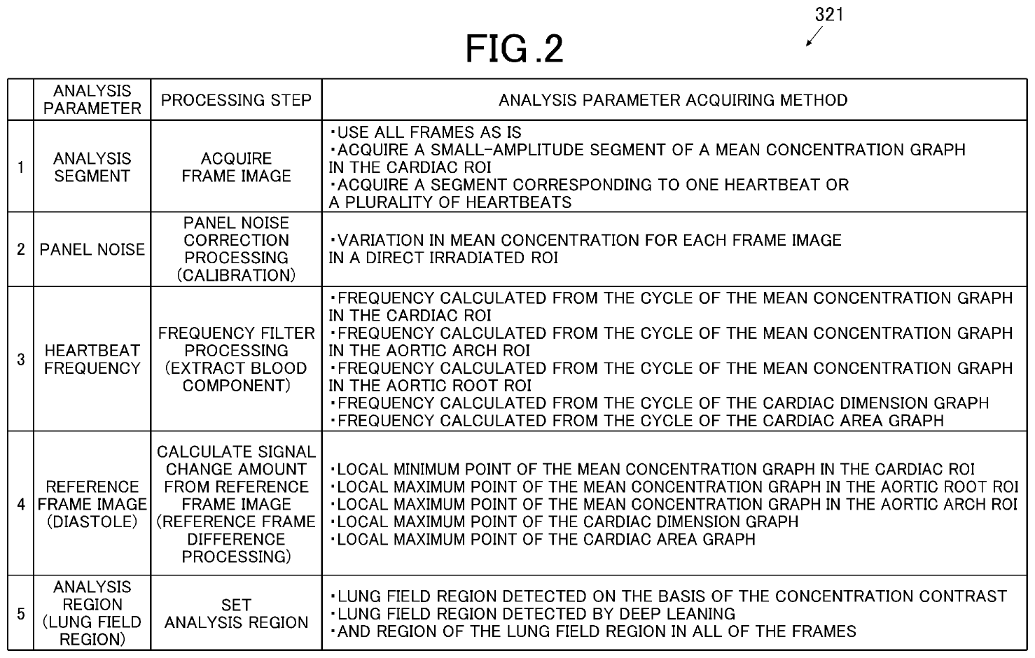

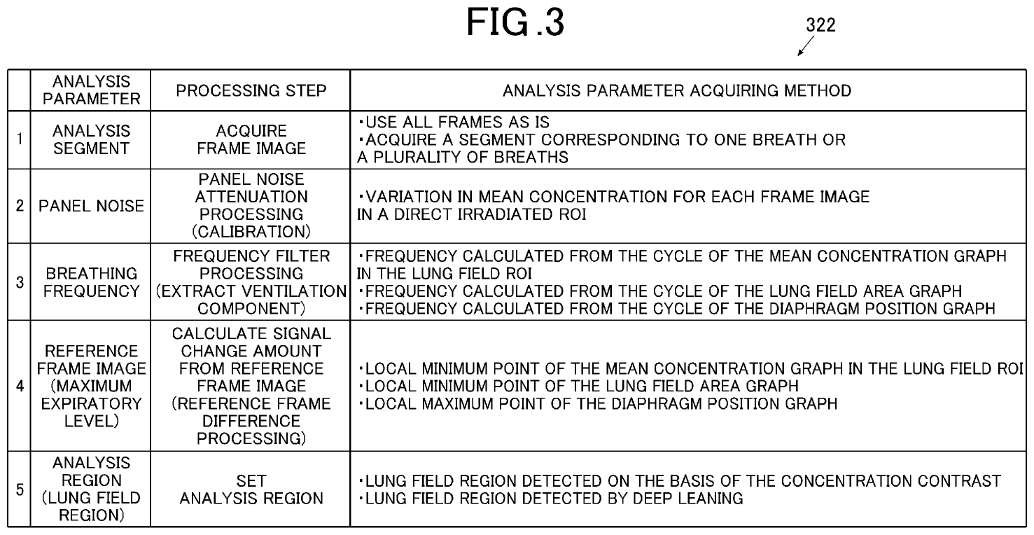

In the present embodiment, the storage section 32 stores an analysis parameter table for each type of analysis processing executable on dynamic images. FIG. 2 is a diagram illustrating an example of the analysis parameter table 321 in a case where the type of analysis processing is blood flow analysis. FIG. 3 is a diagram illustrating an example of an analysis parameter table 322 in a case where the type of analysis processing is ventilation analysis. As shown in FIGS. 2 and 3, in the analysis parameter table, analysis parameters (types of analysis parameters) used in analysis processing, processing steps in which the analysis parameters are used in the analysis processing, and methods of acquiring the analysis parameters are stored in association with each other.

Note that in the present embodiment, the case where a plurality of types of analysis parameters are used in analysis processing will be described as an example. However, one type of analysis parameter may be used in the analysis processing.

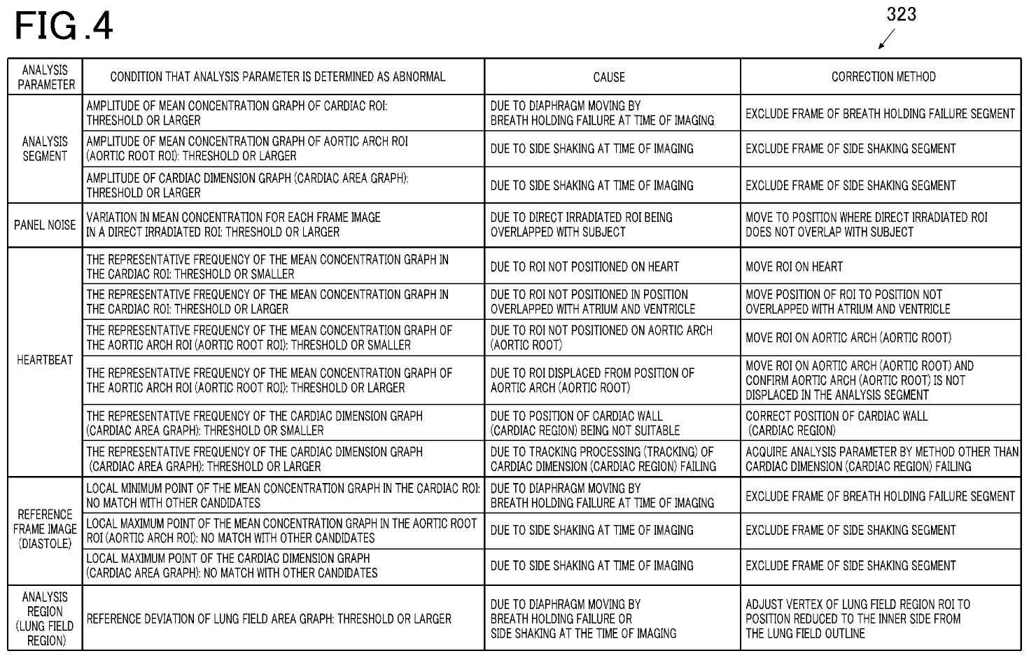

Furthermore, the storage section 32 stores a cause identification table for each type of analysis processing. FIG. 4 is a diagram illustrating an example of the cause identification table 323 in a case where the type of analysis processing is blood flow analysis. As illustrated in FIG. 4, analysis parameters (types of analysis parameters) used in analysis processing, conditions under which the analysis parameters are determined to be abnormal (abnormality conditions), and the causes of abnormality and correction methods are stored in the cause identification table in an associated manner.

The operation part 33 includes a keyboard having cursor keys, number input keys, various function keys, and the like, and a pointing device such as a mouse. The operation part 33 outputs, to the controller 31, an instruction signal input by a user's key operation on the keyboard or a user's mouse operation. Furthermore, the operation part 33 may include a touch screen on the display screen of the display part 34. In this case, the operation part 33 outputs an instruction signal input via the touch screen to the controller 31.

The display part 34 is formed of a monitor such as an LCD or a CRT. The display part 34 performs various displays in accordance with an instruction of a display signal input from the controller 31.

The communication section 35 includes a LAN adapter, a modem, and a TA. The communication section 35 controls transmission and reception of data to and from external devices such as the imaging console 2 and the PACS connected to the communication network NT.

### [Operation of Dynamic Analysis System 100]

Next, an operation of the dynamic analysis system 100 according to the present embodiment will be described.

### (Operation of Imaging Apparatus 1 and Imaging Console 2)

First, imaging operation performed by the imaging apparatus 1 and the imaging console 2 will be described.

FIG. 5 is a flowchart illustrating imaging control processing executed by the controller 21 of the imaging console 2. The imaging control processing is executed by the controller 21 in cooperation with the program stored in the storage section 22.

First, the controller 21 receives an input of patient information and examination information of a subject (subject M) by an operation of the operation part 23 by a photographer (step S1).

The patient information includes information such as a patient ID, name, age, sex, height, and weight. The examination information includes an examination ID, an examination target site, and an analysis type. In the present embodiment, the examination target site is the chest. Furthermore, examples of the type of analysis include blood flow analysis and ventilation analysis.

Next, based on the input patient information and examination information, the controller 21 reads radiation emission conditions from the storage section 22 and sets the conditions in the radiation emission control device 12. In addition, the controller 21 reads the image reading conditions from the storage section 22 and sets the image reading conditions in the reading control apparatus 14 (step S2).

Next, the controller 21 waits for a radiation emission instruction by the operation of the operation part 23 (step S3). Here, the photographer arranges the subject M between the radiation source 11 and the radiation detection section 13 to perform positioning. At the time when the imaging preparation is completed, the photographer operates the operation part 23 to input a radiation emission instruction.

When the radiation emission instruction is input by the operation of the operation part 23 (step S3: YES), the controller 21 outputs an imaging start instruction to the radiation emission control device 12 and the reading control apparatus 14 to start the dynamic imaging (step S4). That is, the controller 21 causes the radiation source 11 to emit radiation at pulse intervals set in the radiation emission control device 12. Further, the controller 21 causes the radiation detection section 13 to acquire a frame image. During the dynamic imaging, the photographer performs breathing guidance such as “inhale”. “exhale”, and “hold the breath” based on the type of analysis included in the examination information. For example, in the case of ventilation analysis, the photographer performs breathing guidance such as “inhale” and “exhale”, and performs imaging in a breathing state. In the case of the blood flow analysis, since the movement of structures due to the breathing becomes noise, the photographer performs breath holding guidance such as “hold the breath” and performs imaging in a breath holding state. Note that the imaging apparatus 1 may include an audio output unit and a display part. Next, when the imaging start instruction is output, the audio output unit of the imaging apparatus 1 may provide a sound or a display of breathing guidance such as “inhale”. “exhale”, and “hold the breath”.

When a radiation emission end instruction is input by the operation of the operation part 23, the controller 21 outputs an imaging end instruction to the radiation emission control device 12 and the reading control apparatus 14 to stop the imaging operation.

The frame images acquired by imaging are sequentially input to the imaging console 2. The controller 21 stores the input frame images in the storage section 22 in association with numbers (frame numbers) indicating the order of capturing (step S5). Further, the controller 21 causes the display part 24 to display the input frame image (step S6). The photographer checks the positioning and the like with the displayed dynamic image, and determines whether an image suitable for diagnosis has been acquired by imaging (imaging OK) or re-imaging is necessary (imaging NG). Then, the photographer operates the operation part 23 to input the determination result.

When the determination result indicating that the imaging is OK is input by a predetermined operation of the operation part 23 (step S7: YES), the controller 21 attaches the additional information to each of a series of frame images acquired by the dynamic imaging and transmits the frame images to the analyzing console 3 via the communication section 25 (step S8). The additional information includes information such as an identification ID for identifying a dynamic image, patient information, examination information, a radiation emission condition, an image reading condition, and a number (frame number) indicating an imaging order. Then, the controller 21 ends the imaging control process.

On the other hand, when the determination result indicating imaging NG is input by a predetermined operation of the operation part 23 (step S7: NO), the controller 21 deletes the series of frame images stored in the storage section 22 (step S9), and ends the imaging control process. In this case, re-imaging is required.

### (Operation of the Analyzing Console 3)

Next, the operation of the analyzing console 3 will be described.

In the analyzing console 3, when a series of frame images of the dynamic image of the chest is received from the imaging console 2 via the communication section 35, a dynamic analysis process A shown in FIG. 6 is executed. The dynamic analysis processing A is executed by the controller 31 in cooperation with the program stored in the storage section 32. Hereinafter, the dynamic analysis processing A will be described with reference to FIG. 6.

First, the controller 31 acquires a dynamic image received by the communication section 35 (step S11).

Next, the controller 31 selects analysis processing (the type of analysis processing) to be performed (step S12).

For example, the controller 31 selects analysis processing based on information on the type of analysis included in the examination information appended to the dynamic image. Examples of the type of analysis processing include blood flow analysis and ventilation analysis.

Next, the controller 31 acquires each of a plurality of types of analysis parameters used in the selected analysis process by a plurality of acquisition methods on the basis of the received dynamic image, and sets the analysis parameters as analysis parameter candidates (step S13).

For example, the controller 31 reads an analysis parameter table corresponding to the selected analysis processing from the storage section 32. The controller 31 acquires each of the plurality of types of analysis parameters used in the selected analysis process designated in the read analysis parameter table by the plurality of acquisition methods designated in the read analysis parameter table.

For example, when the selected analysis process is the blood flow analysis, as shown in FIG. 2, the controller 31 acquires each of five types of analysis parameters of an analysis segment, panel noise, heartbeat frequency, reference frame image, and analysis region based on the dynamic image by a plurality of acquisition methods shown in FIG. 2. In this manner, the controller 31 acquires a plurality of analysis parameter candidates used in the selected analysis process.

For example, when the selected analysis processing is blood flow analysis, the controller 31 acquires an analysis segment by the following methods (A1) to (A3).

### (A1) Use all Frames as is

The controller 31 acquires all the frame images of the received dynamic images as analysis segments.

### (A2) Acquire a Small-Amplitude Segment of a Mean Concentration Graph in the Cardiac ROI

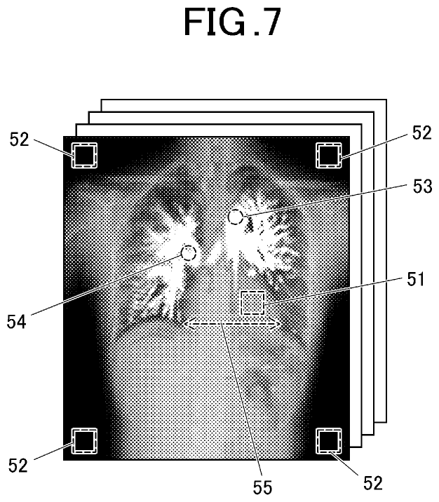

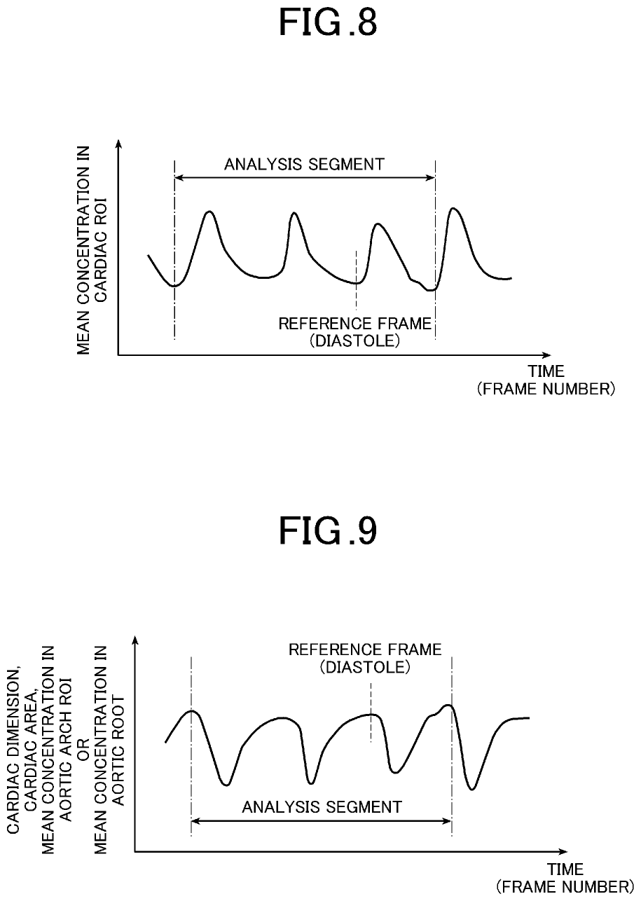

First, the controller 31 extracts the cardiac region from each frame image of the acquired dynamic image. Next, as illustrated in FIG. 7, the controller 31 sets the cardiac ROI 51 in the extracted cardiac region. Next, as shown in FIG. 8, the controller 31 generates a waveform (mean concentration graph) indicating a temporal change in the mean value (mean concentration) of the signal values of the pixels in the cardiac ROI 51. Then, the controller 31 acquires a segment in which the amplitude of the generated waveform is within a predetermined range as an analysis segment.

### (A3) Acquire a Segment Corresponding to One Heartbeat or a Plurality of Heartbeats

First, the controller 31 extracts a cardiac region from each frame image of the acquired dynamic images. Next, as illustrated in FIG. 7, the controller 31 sets the cardiac ROI 51 in the extracted cardiac region. Next, as shown in FIG. 8, the controller 31 generates a waveform (mean concentration graph) indicating a temporal change in the mean value (mean concentration) of the signal values of the pixels in the cardiac ROI 51. Blood is ejected into and ejected from the cardiac region in accordance with heartbeats. Thus, the concentration of the cardiac region decreases or increases. That is, the waveform indicating the temporal change of the concentration in the cardiac region represents the waveform of the heartbeat. Then, the controller 31 acquires, as an analysis segment, a frame image corresponding to a segment of a predetermined cycle (one or more cycles) of the generated waveform.

In (A2) and (A3), the mean concentration graph of the aortic arch ROI 53 shown in FIG. 7 may be used instead of the mean concentration graph of the cardiac ROI 51. Alternatively, instead of the mean concentration graph of the cardiac ROI 51, the mean concentration graph of the aortic root ROI 54 may be used. Alternatively, instead of the mean concentration graph of the cardiac ROI 51, a waveform of a temporal change in a cardiac dimension 55 (cardiac dimension graph) may be used. Alternatively, instead of the mean concentration graph of the cardiac ROI 51, a waveform of a temporal change in cardiac area (cardiac area graph) or the like may be used.

For example, when the selected analysis process is the blood flow analysis, the controller 31 acquires the panel noise by the following method (B1).

(B1) First, the controller 31 extracts a plurality of direct irradiated regions from each frame image of the acquired dynamic image. Next, as shown in FIG. 7, the controller 31 sets a direct irradiated ROI 52 in each of the extracted direct irradiated regions. The direct irradiated region is an image region corresponding to a region of the radiation detection section 13 which is directly irradiated with radiation. That is, the direct irradiated region is a region where the subject does not appear in the frame image. Since the pixel value of the direct irradiated region is much higher than that of other regions, for example, a high-concentration region having a signal value equal to or higher than a predetermined threshold value can be recognized as the direct irradiated region. Next, the controller 31 calculates the variation in the mean value (mean concentration) of the signal values of the pixels for each frame image for each direct irradiated ROI 52. That is, the controller 31 calculates, for each direct irradiated ROI 52, the variation of the mean concentration in the direct irradiated ROI 52 for each frame image. This variation can be obtained, for example, by calculating the difference between the median value or mean value of the mean concentration calculated from all the frame images and the median value or mean value of the mean concentration calculated from each frame image. Next, the controller 31 acquires the calculated variation as panel noise.

For example, when the selected analysis processing is blood flow analysis, the controller 31 acquires heartbeat frequencies by the following methods (C1) to (C5).

(C1) Frequency Calculated from the Mean Concentration Graph of the Cardiac ROI

First, the controller 31 extracts the cardiac region from each frame image of the acquired dynamic image. Next, as illustrated in FIG. 7, the controller 31 sets the cardiac ROI 51 in the extracted cardiac region. Next, as shown in FIG. 8, the controller 31 generates a waveform (mean concentration graph) indicating a temporal change in the mean value (mean concentration) of the signal values of the pixels in the cardiac ROI 51. Next, the controller 31 performs Fourier transform on the generated waveform to calculate a frequency spectrum. Next, when the controller 31 determines that the heart rate falls within, for example, 35 to 220BPM, the controller 31 searches for a frequency (representative frequency) having the maximum intensity among 0.58 to 3.67 Hz and determines the frequency as heartbeat frequency.

(C2) Frequency Calculated from the Mean Concentration Graph in the Aortic Arch ROI

First, the controller 31 extracts a region of the aortic arch from each frame image of the acquired dynamic images. As illustrated in FIG. 7, the controller 31 sets the aortic arch ROI 53 in the extracted region. Next, as shown in FIG. 9, the controller 31 generates a waveform (mean concentration graph) indicating a temporal change in the mean value (mean concentration) of the pixel values in the aortic arch ROI 53. Next, the controller 31 performs Fourier transform on the generated waveform to calculate a frequency spectrum. Next, when the controller 31 determines that the heart rate falls within, for example, 35 to 220BPM, the controller 31 searches for a frequency (representative frequency) having the maximum intensity among 0.58 to 3.67 Hz and determines the frequency as heartbeat frequency.

(C3) Frequency Calculated from the Mean Concentration Graph in the Aortic Root ROI

First, the controller 31 extracts a region of the aortic root from each frame image of the acquired dynamic images. Next, as illustrated in FIG. 7, the controller 31 sets the aortic root ROI 54 in the extracted region. Next, as shown in FIG. 9, the controller 31 generates a waveform (mean concentration graph) indicating a temporal change in the mean value (mean concentration) of the pixel values in the aortic root ROI 54. Next, the controller 31 performs Fourier transform on the generated waveform to calculate a frequency spectrum. Next, when the controller 31 determines that the heart rate falls within, for example, 35 to 220BPM, the controller 31 searches for a frequency (representative frequency) having the maximum intensity among 0.58 to 3.67 Hz and determines the frequency as heartbeat frequency.

Here, the aortic arch and the aortic root are regions in which the concentration changes according to the blood flow due to the heartbeat.

(C4) Frequency Calculated from the Cardiac Dimension Graph

First, the controller 31 extracts the cardiac region from each frame image of the acquired dynamic image. Next, as illustrated in FIG. 7, the controller 31 acquires the maximum value of the diameter (width) of the extracted cardiac region as a cardiac dimension 55. Next, as shown in FIG. 9, the controller 31 generates a waveform (cardiac dimension graph) indicating a temporal change in the cardiac dimension 55. Next, the controller 31 performs Fourier transform on the generated waveform to calculate a frequency spectrum. Next, when the controller 31 determines that the heart rate falls within, for example, 35 to 220BPM, the controller 31 searches for a frequency (representative frequency) having the maximum intensity among 0.58 to 3.67 Hz and determines the frequency as heartbeat frequency.

(C5) Frequency Calculated from the Cardiac Area Graph

First, the controller 31 extracts the cardiac region from each frame image of the acquired dynamic image. Next, as illustrated in FIG. 7, the controller 31 calculates the area of the extracted cardiac region. Next, as shown in FIG. 9, the controller 31 generates a waveform (heart area graph) indicating a temporal change in the heart area. Next, the controller 31 performs Fourier transform on the generated waveform to calculate a frequency spectrum. Next, when the controller 31 determines that the heart rate falls within, for example, 35 to 220BPM, the controller 31 searches for a frequency (representative frequency) having the maximum intensity among 0.58 to 3.67 Hz and determines the frequency as heartbeat frequency.

For example, when the selected analysis processing is blood flow analysis, the controller 31 acquires the reference frame image by the following methods (D1) to (D5).

(D1) The controller 31 acquires, as the reference frame image, the frame image (frame number) corresponding to the local minimum point of the waveform indicating the temporal change of the mean concentration in the cardiac ROI 51.

(D2) The controller 31 acquires, as the reference frame image, a frame image (frame number) corresponding to the local maximum point of the waveform indicating the temporal change of the mean concentration in the aortic root ROI 54.

(D3) The controller 31 acquires, as the reference frame image, the frame image (frame number) corresponding to the local maximum point of the waveform indicating the temporal change of the mean concentration in the aortic arch ROI 53.

(D4) The controller 31 acquires, as the reference frame image, the frame image (frame number) corresponding to the local maximum point of the waveform indicating the temporal change of the mean concentration in the cardiac dimension 55.

(D5) The controller 31 acquires, as the reference frame image, the frame image (frame number) corresponding to the local maximum point of the waveform indicating the temporal change in the area of the cardiac region.

For example, when the selected analysis processing is blood flow analysis, the controller 31 acquires an analysis region by any of the following methods (E1) to (E3).

(E1) The controller 31 detects the lung field region from each frame image on the basis of the concentration contrast and acquires, as the analysis region, the lung field region in the frame image having the smallest lung field area or the frame image in diastole.

(E2) The controller 31 detects the lung field region from each frame image by Deep Leaning, and acquires, as the analysis region, the lung field region in the frame image with the smallest lung field area or the frame image in the diastole.

(E3) The controller 31 detects the lung field region from each frame image on the basis of the concentration contrast or by Deep Leaning, and acquires an AND region of the lung field regions of all the frame images as the analysis region.

In a case where the selected analysis process is ventilation analysis, the controller 31 acquires the plurality of analysis parameter candidates for each of the plurality of types of analysis parameters on the basis of an analysis parameter table 322 illustrated in FIG. 3.

Next, the controller 31 evaluates whether the analysis parameter candidates are within an appropriate range, and excludes analysis parameter candidates that are not within the appropriate range from the calculation targets of the reliability (step S14).

Here, in the storage section 32, the appropriate range of each analysis parameter is stored in advance for each type of analysis. For example, in a case where the selected analysis processing is blood flow analysis, the respective appropriate ranges of the analysis segment, the panel noise, the heartbeat frequency, the reference frame image, and the analysis region can be set to the following ranges.

analysis segment: minimum number of frames required for processing=frame image segment of 6 seconds or more

panel noise: a variation in mean concentration of the direct irradiated ROI in each frame image is within 1%

heartbeat frequencies: 0.58 to 3.67 Hz (35 to 220BPM)

reference frame image: frame number is within a range of 1 to the maximum number of frames

analysis region: region size is ¼ to ¾ of image size

Next, the controller 31 calculates, for each analysis parameter (type of analysis parameter), the reliability of each analysis parameter candidate on the basis of the number of times the value of each of the plurality of analysis parameter candidates matches the value of any other analysis parameter candidate (step S15).

The reliability of each analysis parameter candidate is a value indicating the degree to which each analysis parameter is reliable.

Here, analysis parameter candidates for which the difference is within a range defined in advance for each type of analysis parameter are considered to match.

The reliability (%) can be calculated by, for example, the following (Equation 1).

Reliability=(the number of other analysis parameter candidates whose values match those of the analysis parameter candidate whose reliability is to be the target to be calculated+1)=(the total number of analysis parameter candidates) . . . (Equation 1).

Here, for the panel noise, the maximum value among the variations calculated for the respective frame images is used as the analysis parameter candidate used in calculating the reliability.

Note that the total number of analysis parameter candidates may include the number of analysis parameters excluded in step S14.

That is, the analysis parameter candidate having the larger number of other analysis parameter candidates having matching values has a higher reliability.

Here, a method of calculating the reliability in step S15 will be described with calculation of the reliability of the analysis parameter candidate of the heartbeat frequency as an example. Note that in order to simplify the description, the analysis parameter candidates of the heartbeat frequency will be described as the following analysis parameter candidate 1 to analysis parameter candidate 3.

analysis parameter candidate 1: the representative frequency of the mean concentration graph in the cardiac ROI 51=1.5 Hz analysis parameter candidate 2: the representative frequency of the mean concentration graph in the aortic arch ROI 53=1.0 Hz analysis parameter candidate 3: the representative frequency of the temporal change graph of the cardiac dimension 55=1.0 Hz

When the values of the analysis parameter candidate 1 to the analysis parameter candidate 3 are compared, the analysis parameter candidate 2 and the analysis parameter candidate 3 match, but the analysis parameter candidate 1 does not match the other parameter candidates. Therefore, from (Equation 1), the reliability of each of the analysis parameter candidate 1 to the analysis parameter candidate 3 is as follows.

Reliability of analysis parameter candidate 1=1/3=33.3 (%)

Reliability of analysis parameter candidate 2=2/3=66.7 (%)

Reliability of analysis parameter candidate 3=2/3=66.7 (%)

In addition, priority may be set in advance in the acquisition method of the analysis parameter candidate, and the reliability may be calculated by weighting the analysis parameter candidate based on the priority.

For example, assuming that the weighting coefficient of the above-described analysis parameter candidate 1 is 1.5, the weighting coefficient of the analysis parameter candidate 2 is 1.2, and the weighting coefficient of the analysis parameter candidate 3 is 0.9, the reliability of each analysis parameter candidate can be calculated as follows.

Reliability of analysis parameter candidate 1=1×1.5/(1×1.5+1×1.2+1×0.9)=41.7 (%) Reliability of analysis parameter candidate 2=(1×1.2+1×0.9)/(1×1.5+1×1.2+1×0.9)=58.3 (%) Reliability of analysis parameter candidate 3=(1×1.2+1×0.9)/(1×1.5+1× 1.2+1×0.9)=58.3 (%)

Next, based on the calculated reliability, the controller 31 determines analysis parameters to be used for analysis processing (step S16).

The controller 31 determines, for each type of analysis parameter to be used in analysis processing, the analysis parameter candidate having the highest calculated reliability as the analysis parameter to be used. For example, in the example of the heartbeat frequencies described above, the controller 31 determines 1.0 Hz, which is the value of the analysis parameter candidate 2 and the analysis parameter candidate 3, as the analysis parameter of the heartbeat frequency. The controller 31 may cause the display part 34 to display the determined analysis parameters so that the user can check them.

Note that when the degrees of reliability of the analysis parameter candidates that do not match are equal, the controller 31 determines the analysis parameter to be employed, on the basis of the priority of the method of acquiring the analysis parameter candidate described above. Alternatively, the controller 31 may display the analysis parameter candidates on the display part 34 for the user to select.

Next, the controller 31 executes the analysis processing selected in step S12 using the determined analysis parameters to generate an analysis result image (step S17).

For example, when the selected analysis processing is blood flow analysis, the controller 31 first acquires frame images of the determined analysis segment from the received dynamic images. Next, the controller 31 applies noise correction processing to each frame image on the basis of the determined value of the panel noise. For example, the controller 31 subtracts or adds the variation of the mean concentration of the directly irradiated ROI 52 in each frame image from or to the signal value of each frame image, and corrects the signal value to a value close to the median value (or mean value) of all the frame images. Next, the controller 31 sets the determined analysis region in each frame image as the analysis region, and divides the set analysis region into block regions of a predetermined size (for example, 10 mm×10 mm). Next, the controller 31 replaces the signal value (concentration value) of each pixel in the block region with the representative value (e.g., mean value) of the signal values in the block region and smoothes the signal values. Next, the controller 31 extracts a blood flow component by performing high-pass filtering on the inside of the analysis region with the determined heartbeat frequency as a cutoff frequency. Next, for each frame image, the controller 31 calculates, for each block region of the analysis region, a difference value (reference frame difference value) of the signal value from the corresponding block region of the determined reference frame image. The corresponding block region is a block region having the same position in the image. Then, the controller 31 adds a color corresponding to the inter-frame difference value to each block region of each frame image to generate an analysis result image representing the blood flow volume of each block region.

Furthermore, for example, if the selected analysis processing is ventilation analysis, the controller 31 first acquires frame images of the determined analysis segment from the received dynamic images. Next, the controller 31 applies noise correction processing to each frame image on the basis of the determined value of the panel noise. For example, the controller 31 subtracts or adds the variation of the mean concentration of the directly irradiated ROI 52 in each frame image from or to the signal value of each frame image, and corrects the signal value to a value close to the median value (or mean value) of all the frame images. Next, the controller 31 sets the determined analysis region in each frame image as the analysis region, and divides the set analysis region into block regions of a predetermined size (for example, 10 mm×10 mm). Next, the controller 31 replaces the signal value (concentration value) of each pixel in the block region with the representative value (e.g., mean value) of the signal values in the block region and smoothes the signal values. Next, the controller 31 extracts a ventilation component by applying low-pass filter processing with the determined breathing frequency as a cutoff frequency in the analysis region. Next, for each frame image, the controller 31 calculates, for each block region of the analysis region, a difference value (reference frame difference value) of the signal value from the corresponding block region of the determined reference frame image. Then, the controller 31 adds a color corresponding to the inter-frame difference value to each block region of each frame image to generate an analysis result image representing the ventilation amount of each block region.

Next, the controller 31 allows the display part 34 to display the reliability of each analysis parameter used in the analysis (step S18).

Next, the controller 31 determines whether the reliability of each analysis parameter used in the analysis processing is higher than or equal to a predetermined threshold value (step S19).

In a case where it is determined that the reliability of each analysis parameter used in the analysis process is equal to or greater than the predetermined threshold value (Step S19: YES), the controller 31 determines that it is not necessary to correct the analysis parameter. Then, the controller 31 stores the received dynamic image and analysis result image in the storage section 32 (step S20), and ends the dynamic analysis process A.

On the other hand, in a case where it is determined that there is an analysis parameter of which the reliability is less than the predetermined threshold value (Step S19; NO), the controller 31 determines that the analysis parameter needs to be corrected. Then, the controller 31 performs a process of specifying a cause (cause of low reliability) for which correction is necessary and a correction method for each of the analysis parameters of which the reliabilities are less than the predetermined threshold values (step S21).

For example, the controller 31 refers to the cause identification table (refer to FIG. 4) stored in the storage section 32, and determines whether the analysis parameter of which the reliability is less than a predetermined threshold value corresponds to the abnormality condition of the cause identification table. When determining that the abnormality condition is satisfied, the controller 31 identifies the cause and the correction method associated with the abnormality condition as the cause and the correction method that needs to be corrected.

Next, the controller 31 causes the display part 34 to display the analysis parameter correction notification screen 341 (step S22). The analysis parameter correction notification screen 341 provides notification of an analysis parameter that requires correction, and receives correction of the analysis parameter by the user.

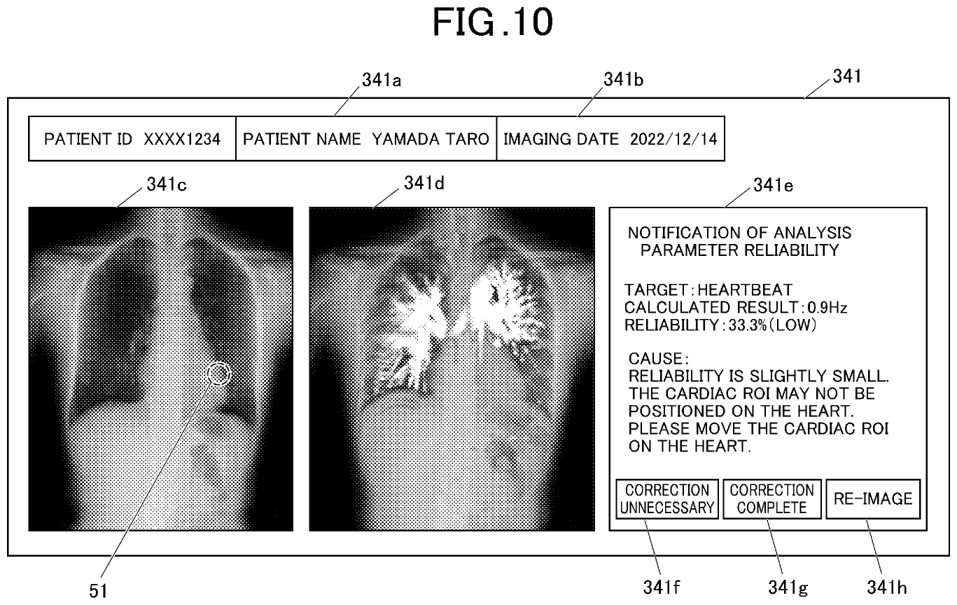

FIG. 10 is a diagram illustrating an example of an analysis parameter correction notification screen 341. FIG. 10 illustrates, as an example, a screen for providing notification of correction of the heartbeat frequency. As shown in FIG. 10, patient information 341a, an imaging date 341b, a correction input field 34 lc, an analysis result image 341d, notification information 341e, and the like are displayed on the analysis parameter correction notification screen 341.

The correction input field 341c is a field for the user to input correction information for correcting the analysis parameter. In the correction input field 341c, a captured image (dynamic image) and a user interface (UI) for a user to input correction information for correcting an analysis parameter are displayed.

For example, as illustrated in FIG. 10, image information used for acquiring analysis parameters is highlighted on the captured image (dynamic image) in the correction input field 341c. The image information used for acquiring the analysis parameters includes, for example, an ROI, a measurement position such as a cardiac dimension, and a measurement region such as an outline and feature points of a cardiac region. FIG. 10 illustrates a case where the cardiac ROI 51 is highlighted with a circle. The ROI, the outline, the feature points, and the like highlighted on the captured image in the correction input field 341c can be corrected, such as moved or enlarged/reduced, by a user's operation on the operation part 33. Note that, for example, in the case of an analysis parameter that is difficult to correct on a captured image, such as an analysis segment, a UI may be displayed separately from the captured image. For example, when the analysis segment needs to be corrected, a graph as illustrated in FIG. 8 or 9 may be displayed so that the user can set the start point and the end point of the analysis segment on the graph by operating the operation part 33. In addition, options of the method of acquiring the analysis parameter may be displayed, and the method of acquiring the analysis parameter may be corrected according to the operation of the operation part 33 by the user.

The analysis result image 341d is an analysis result image generated by the analysis processing in step S17. Note that the analysis result image 341d may be displayed such that a moving image can be played back, or any one of the frame images of the analysis result images may be displayed.

The notification information 341e is information for providing a notification that the analysis parameter needs to be corrected. The notification information 341e includes, for example, the type of analysis parameter to be corrected, the acquisition result (value) of the analysis parameter, the reliability, the cause of low reliability, and the correction method.

A correction unnecessary button 341f, a correction completion button 341g, and a re-imaging button 341h are displayed in the vicinity of the notification information 341c.

The correction unnecessary button 341f is a button for inputting that correction is unnecessary. The correction completion button 341g is a button for inputting, from the correction input field 341c, that correction of the information used for acquisition of the analysis parameters has been completed. The re-imaging button 341h is a button for inputting that re-imaging is to be performed.

The user can easily recognize the analysis parameter that needs to be corrected by checking the notification information 341e displayed on the analysis parameter correction notification screen 341. In addition, it is possible to check the cause of the necessity of correction and the correction method and to easily grasp how the correction should be performed to improve the reliability of the analysis parameters.

Next, the controller 31 determines whether the correction completion button 341g has been pressed (step S23).

When determining that the correction completion button 341g has been pressed (step S23: YES), the controller 31 reacquires (corrects) the analysis parameter to be corrected, based on the correction information input from the correction input field 341c (step S24).

In step S24, in a case where the correction of the acquisition method of the analysis parameter is not input from the correction input field 341c, the controller 31 re-acquires the analysis parameter of the correction target in consideration of the correction content by the same acquisition method as the analysis parameter determined in step S16. For example, when the position of the cardiac ROI is corrected, the controller 31 reacquires (corrects) the analysis parameter of the correction target by the same acquisition method as the analysis parameter determined in step S16 using the cardiac ROI after the correction. On the other hand, in a case where the correction of the acquisition method of the analysis parameter is input by the user, the controller 31 reacquires (corrects) the analysis parameter of the correction target by the acquisition method after the correction.

Next, the controller 31 executes again (re-analyzes) the analysis processing selected in step S12 on the received dynamic images using the analysis parameters (step S25). The controller 31 executes the analysis process selected in step S12 on the received dynamic image again by using the re-acquired analysis parameter for the analysis parameter as the target to be corrected. Using the analysis parameters determined in step S16 for the other analysis parameters, the controller 31 executes again the analysis processing selected in step S12 on the received dynamic images. Then, the controller 31 stores the received dynamic image and the analysis result image generated by the re-analysis in the storage section 32 (step S26), and ends the dynamic analysis process A.

On the other hand, if the controller 31 determines that the correction completion button 341g has not been pressed (step S23: NO), the controller 31 determines whether a correction unnecessary button 341f has been pressed (step S27).

In a case where it is determined that the correction unnecessary button 341f is pressed (step S27: YES), the controller 31 proceeds to step S20, stores the received dynamic image and the analysis result image generated in step S17 in the storage section 32 (step S20), and ends the dynamic analysis process A.

If the controller 31 determines that the correction unnecessary button 341f has not been pressed (step S27: NO), the controller 31 determines whether the re-imaging button 341h has been pressed (step S28). If the controller 31 determines that the re-imaging button 341h has not been pressed (step S28: NO), the controller 31 returns the process to step S23.

In a case where it is determined that the re-imaging button 341h is pressed (step S28: YES), the controller 31 ends the dynamic analysis process A.

The user performs re-imaging of the subject M with the imaging apparatus 1. The re-captured dynamic image is transmitted to the analyzing console 3 via the imaging console 2, and the dynamic analysis processing A is performed on the re-captured dynamic image.

The controller 31 transmits the dynamic images and analysis result images stored in the storage section 32 to a PACS (not illustrated) connected to the communication network NT, for example, via the communication section 35. Thus, the dynamic image and the analysis result image are provided for diagnosis by a physician.

As described above, the controller 31 of the analyzing console 3 determines the necessity of the correction of one or a plurality of types of analysis parameters used for the analysis of the acquired dynamic image, and notifies the analysis parameter determined to be corrected by displaying the analysis parameter on the analysis parameter correction notification screen 341. Therefore, the user can easily recognize the analysis parameter that needs to be corrected among the analysis parameters for analyzing the dynamic image. In addition, on the analysis parameter correction notification screen 341, the cause of the necessity of the correction of the analysis parameter and the correction method are notified. Therefore, it becomes possible for the user to easily identify and correct the cause of the necessity of the correction of the analysis parameters and the correction method and to obtain an analysis result image re-analyzed using the corrected analysis parameters.

### <Second Embodiment>

Next, a second embodiment of the present invention will be described.

In the second embodiment, an example will be described in which the reliability of a corrected analysis parameter is calculated, and a notification prompting re-imaging is provided in a case where the reliability of the corrected analysis parameter is lower than a predetermined threshold value.

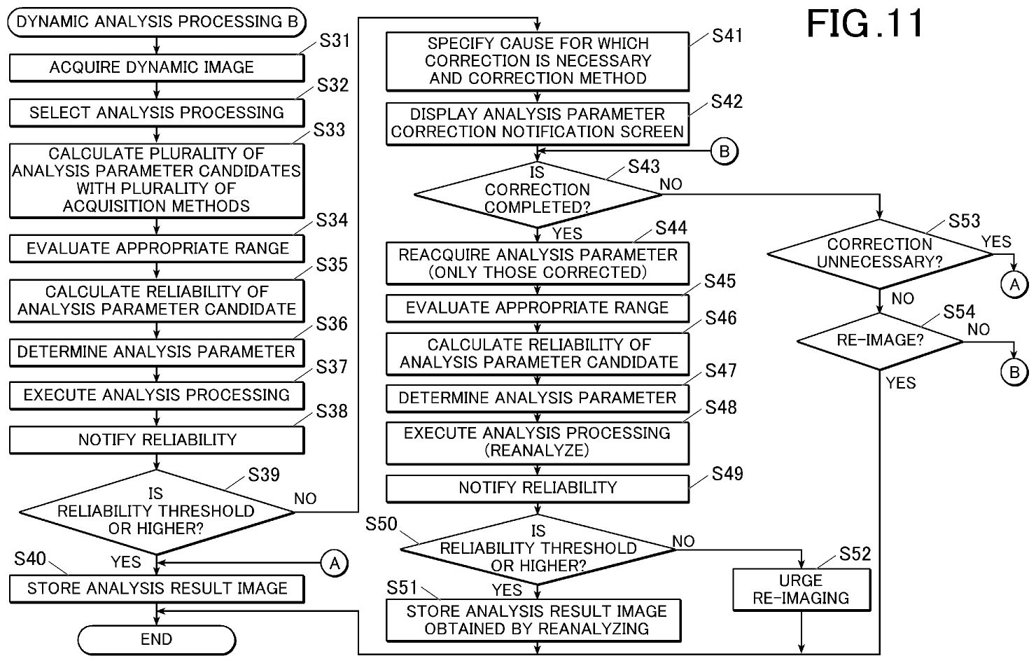

In the second embodiment, the storage section 32 stores a program for the controller 31 to execute dynamic analysis processing B (see FIG. 11) described later. The other constituent elements of the dynamic analysis system 100 are the same as those described in the first embodiment, and hence the description thereof is cited. Hereinafter, operations according to the second embodiment will be described.

In the dynamic analysis processing B, first, the controller 31 executes processing in steps S31 to S42. The processing of steps S31 to S42 is the same as the processing of steps S11 to S22 in FIG. 6, and thus the description thereof will be cited. Note that in the second embodiment, no option for correcting the method of acquiring analysis parameters is displayed on the analysis parameter correction notification screen 341.

Next, in step S43, the controller 31 determines whether the correction completion button 341g has been pressed (step S+3).

In a case where it is determined that the correction completion button 341g is pressed (step S43: YES), the controller 31 reacquires the analysis parameter candidate of the analysis parameter of the correction target based on the correction information input from the correction input field 341c (step S+4).

In step S44, similar to step S13 of FIG. 6, the controller 31 acquires, based on the analysis parameter table stored in the storage section 32, a plurality of analysis parameter candidates for an analysis parameter to be corrected, by a plurality of different acquiring methods.

Next, for each of the plurality of re-acquired analysis parameter candidates, the controller 31 evaluates the appropriate range and calculates the reliability of the analysis parameter candidate, and determines the analysis parameter from among the plurality of analysis parameter candidates (steps S45 to S47). Since processing in steps S45 to S47 is the same as that described in steps S14 to S16 of FIG. 6, the description is cited.

Next, the controller 31 executes again (re-analyzes) the analysis processing selected in step S32 on the received dynamic images using the analysis parameter (step S48). The controller 31 executes again the analysis processing selected in step S32 on the received dynamic images using the re-determined analysis parameters as the analysis parameters as the target to be corrected. The controller 31 executes again (re-analyzes) the analysis processing selected in step S36 on the received dynamic images using the analysis parameters determined in step S32 for the other analysis parameters.

Next, the controller 31 causes the display part 34 to display the reliability of the corrected analysis parameter (step S49).

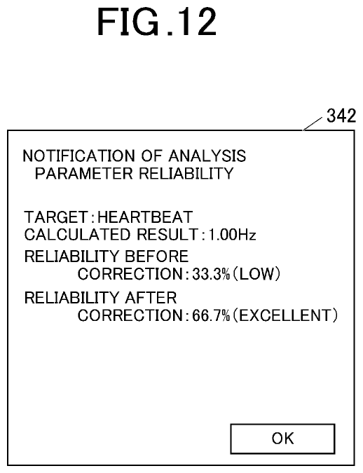

FIG. 12 is a diagram showing an example of the reliability notification screen 342 displayed in step S49. As illustrated in FIG. 12, for example, the type of the corrected analysis parameter, the acquisition result (value), the reliability before correction, and the reliability after correction are displayed on the reliability notification screen 342.

Next, the controller 31 determines whether the corrected reliability is higher than or equal to a predetermined threshold value (step S50).

In a case where it is determined that the corrected reliability is equal to or greater than the predetermined threshold value (step S50: YES), the controller 31 stores the received dynamic image and the analysis result image generated by the re-analysis in the storage section 32 (step S51), and ends the dynamic analysis process B.

When it is determined that the corrected reliability is not equal to or greater than the predetermined threshold value (step S50; NO), the controller 31 causes the display part 34 to display a notification prompting re-imaging (step S52), and ends the dynamic analysis process B.

For example, the controller 31 causes the reliability notification screen 342 to additionally display a notification prompting re-imaging.

On the other hand, in step S43, when it is determined that the correction completion button 341g is not pressed (step S+3: NO), the controller 31 determines whether the correction unnecessary button 341f is pressed (step S53).

In a case where it is determined that the correction unnecessary button 341f is pressed (step S53: YES), the controller 31 proceeds to step S40 and stores the received dynamic image and the analysis result image generated in step S37 in the storage section 32 (step S40). Next, the controller 31 ends the dynamic analysis processing B.

If the controller 31 determines that the correction unnecessary button 341f has not been pressed (step S53: NO), the controller 31 determines whether the re-imaging button 341h has been pressed (step S54).

If the controller 31 determines that the re-imaging button 341h has not been pressed (step S54: NO), the controller 31 returns the processing to step S43.

In a case where it is determined that the re-imaging button 341h is pressed (Step S54: YES), the controller 31 ends the dynamic analysis process B.

The user performs re-imaging of the subject M with the imaging apparatus 1. The re-captured dynamic image is transmitted to the analyzing console 3 via the imaging console 2, and the dynamic analysis processing B is performed on the re-captured dynamic image.

The controller 31 transmits the dynamic images and analysis result images stored in the storage section 32 to a PACS (not illustrated) connected to the communication network NT, for example, via the communication section 35. Thus, the dynamic image and the analysis result image are provided for diagnosis by a physician.

According to the second embodiment, the controller 31 calculates the reliability of the corrected analysis parameter, and provides a notification prompting re-imaging if the reliability of the corrected analysis parameters is lower than a predetermined threshold value. Therefore, in a case where the reliability is low even if the analysis parameter is corrected, it is possible to prompt the user to perform re-imaging.

As described above, the controller 31 of the analyzing console 3 determines the necessity of correction of one or a plurality of types of analysis parameters used for the analysis of the acquired dynamic image, and notifies the analysis parameter determined to be corrected.

Therefore, it is possible for the user to easily recognize the analysis parameter which needs to be corrected among the analysis parameters for analyzing the dynamic image.

For example, the controller 31 calculates the reliability of the analysis parameter and determines, based on the calculated reliability, whether the analysis parameter needs to be corrected. For example, the controller 31 determines that an analysis parameter whose calculated reliability is smaller than a predetermined threshold value needs to be corrected.

Therefore, it is possible to prompt the user to correct an analysis parameter with low reliability.

In addition, the controller 31 acquires a plurality of analysis parameter candidates which are candidates of the analysis parameter by a plurality of different methods, and calculates the number of times the value of each of the plurality of acquired analysis parameter candidates matches the value of another analysis parameter candidate. Next, the controller 31 calculates the reliability of each of the analysis parameter candidates based on the calculated number, and determines the analysis parameter candidate having the highest calculated reliability as the analysis parameter.

Therefore, it is possible to perform analysis using an analysis parameter candidate having the highest reliability among a plurality of analysis parameter candidates as an analysis parameter.

Furthermore, the controller 31 excludes, from the calculation targets of the reliability, analysis parameter candidates that are not within a predetermined appropriate range among the analysis parameter candidates.

Therefore, it is possible to avoid an analysis parameter candidate that is not within the appropriate range from being determined as the analysis parameter.

In addition, the controller 31 notifies the cause of the necessity of the correction of the analysis parameter and the correction method. Therefore, the user can easily recognize how to correct the analysis parameter.

In addition, the controller 31 displays the analysis parameter correction notification screen 341 on the display part 34 and corrects the analysis parameter which is determined to be necessary to be corrected based on the input from the analysis parameter correction notification screen 341. Next, the controller 31 re-analyzes the dynamic image using the corrected analysis parameters and generates an analysis result image again.

Therefore, it is possible to provide an analysis result image using the corrected analysis parameters.

Furthermore, the controller 31 calculates the reliability of the corrected analysis parameters and, if the reliability of the corrected analysis parameters is lower than a predetermined threshold value, provides a notification prompting re-imaging.

Therefore, when the reliability is low even if the analysis parameter is corrected, the user can be prompted to perform re-imaging.

Note that the description in the above embodiment is a preferred example of the present invention, and the present invention is not limited to this.

For example, in the above-described embodiment, the analysis parameter candidate having the highest reliability is determined as the analysis parameter to be used for the analysis from the analysis parameter candidates obtained by calculating the analysis parameter by a plurality of different methods, but the invention is not limited thereto. For example, the analysis parameter may be determined on the basis of a predetermined priority or on the basis of setting. Next, the controller 31 may calculate the reliability of the determined analysis parameter on the basis of the number of analysis parameters whose values match those of the determined analysis parameter among a plurality of analysis parameters that are of the same type as the determined analysis parameter and that are acquired by a method different from the method by which the determined analysis parameter is acquired. For example, the reliability of the determined analysis parameter may be calculated by (Equation2).

Reliability=(the number of analysis parameters whose values match those of the determined analysis parameter, out of a plurality of analysis parameters that are of the same type as the determined analysis parameter and that are acquired by different methods+1)+(the number of analysis parameters of the same type as the determined analysis parameter+1) . . . (Equation 2).

Here, analysis parameter candidates for which the difference is within a range defined in advance for each analysis parameter (type of analysis parameter) are considered to have matching values.

The appropriate range and the threshold value of an analysis parameter or an analysis parameter candidate may be adjustable on the basis of information on the subject, for example, information on the height, the weight, and the like.

Furthermore, the controller 31 may display, on the analysis parameter correction notification screen 341, the reliability of the analysis parameter around the image information used to obtain the analysis parameter.

In addition, in a case where the reliability of the analysis parameter is low: the controller 31 may notify that the reliability of the analysis parameter is low by a sentence.

In addition, the controller 31 may display a warning message on the display part 34 or the display part of the PACS when the analysis result image in which the reliability of the analysis parameter is lower than the predetermined threshold value is transmitted to the PACS or stored.

In addition, in the above-described embodiment, the controller 31 determines the necessity of the correction of the analysis parameter based on the reliability of the analysis parameter, but the determination of the necessity of the correction of the analysis parameter is not limited to the determination based on the reliability.

Furthermore, the method of calculating the reliability is not limited to the above-described method.

Furthermore, in the above-described embodiment, the case where the present invention is applied to an analysis parameter used when an analysis result image is generated from a dynamic image of a chest part has been described as an example, but the present invention may be applied to an analysis parameter used when an analysis result image is generated from a dynamic image of another part.

Furthermore, the analysis processing and the types of analysis parameters described in the above embodiment are merely examples, and are not limited to those described above.

Further, in the above description, an example in which a hard disk, a semiconductor nonvolatile memory, or the like is used as a computer-readable medium of the program according to the present invention has been disclosed, but the present invention is not limited to this example. As another computer-readable medium, a portable recording medium such as a CD-ROM can be applied. In addition, a carrier wave is also applied as a medium for providing data of the program according to the present invention via a communication line.

In addition, the detailed configuration and detailed operation of each device constituting the dynamic analysis system can also be appropriately changed without departing from the spirit and scope of the present invention.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese Patent Application No. 2023-060054, filed on Apr. 3, 2023, including description, claims, drawings and abstract is incorporated herein by reference.

What is claimed is:

1. A dynamic image analysis apparatus comprising:
a hardware processor,
wherein the hardware processor,
acquires a dynamic image obtained by radiographing a dynamic state of a subject,
analyzes the dynamic image using one or more types of analysis parameters,
generates an analysis result image,
determines whether the analysis parameter needs to be corrected, and
notifies the analysis parameter determined to require correction.

2. The dynamic image analysis apparatus according to claim 1, wherein the hardware processor, calculates reliability of the analysis parameter, and
determines a necessity of correction of the analysis parameter based on the calculated reliability.

3. The dynamic image analysis apparatus according to claim 2, wherein the hardware processor determines that the analysis parameter whose calculated reliability is lower than a predetermined threshold value needs to be corrected.

4. The dynamic image analysis device according to claim 2, wherein the hardware processor calculates the reliability of the analysis parameter based on a number of analysis parameters matching the analysis parameter among a plurality of analysis parameters of the same type as the analysis parameter acquired by a method different from the method for acquiring the analysis parameter.

5. The dynamic image analysis apparatus according to claim 2, wherein the hardware processor,
acquires a plurality of analysis parameter candidates serving as candidates for the analysis parameter by a plurality of different methods, and calculates the reliability of each of the analysis parameter candidates based on a number each of the plurality of acquired analysis parameter candidates matches another of the analysis parameter candidates, and
determines the analysis parameter candidate having the highest calculated reliability as the analysis parameter.

6. The dynamic image analysis apparatus according to claim 5, wherein the hardware processor excludes, from a target used to calculate the reliability, the analysis parameter candidate that is not within a predetermined appropriate range among the analysis parameter candidates.

7. The dynamic image analysis apparatus according to claim 1, wherein the hardware processor further notifies a cause of necessity of correction of the analysis parameter and a correction method.

8. The dynamic image analysis apparatus according to claim 1, wherein the hardware processor,
receives, as an input from a user, correction information for correcting the analysis parameter determined to require correction,
corrects, based on the input from the user, the analysis parameter determined to require correction, and
re-analyzes the dynamic image using the corrected analysis parameter and generates an analysis result image again.

9. The dynamic image analysis apparatus according to claim 8, wherein the hardware processor calculates reliability of the corrected analysis parameter, and in a case where the reliability of the corrected analysis parameter is smaller than a predetermined threshold value, provides a notification prompting re-imaging.

10. A non-transitory computer-readable recording medium including a program for causing a computer used in a dynamic image analysis apparatus to,
acquire a dynamic image obtained by radiographing a dynamic state of a subject,
analyze the dynamic image using one or more types of analysis parameters,
generate an analysis result image,
determine whether the analysis parameter needs to be corrected, and
notify the analysis parameter determined to require correction.

11. A dynamic image analysis method comprising:
acquiring a dynamic image obtained by radiographing a dynamic state of a subject;
analyzing the dynamic image using one or more types of analysis parameters;
generating an analysis result image;
determining whether the analysis parameter needs to be corrected; and
notifying the analysis parameter determined to require correction.
